(19) United States
(12) Patent Application Publication
(10) Pub. No.: US 2007/0082406 A1
(43) Pub. Date: Apr. 12, 2007

(54) Method for determining the concentration of acrylic acid-c1-c8-ester in combustible gas

(76) Inventors: Gerd Mansfeld, Eschershausen (DE); Dirk Müller, Dassel (DE)

Correspondence Address: ROYLANCE, ABRAMS, BERDO & GOODMAN, L.L.P., 1300 19TH STREET, N.W., SUITE 600, WASHINGTON,, DC, 20036, US

(21) Appl. No.: 10/570,518

(22) PCT Filed: Aug. 30, 2004
(86) PCT No.: PCT/EP04/51955

§ 371 Date: Oct. 10, 2006

(30) Foreign Application Priority Data
Sep. 5, 2003 (DE) 103 41 400.2

(51) Int. Cl. G01N 33/00

(52) U.S. Cl. 436/128; 436/129

(57) ABSTRACT

The present invention describes a method for the determination of the concentration of acrylic acid C₁-C₈ esters in fuel gas, with the following step:
bringing the fuel gas containing acrylic acid C₁-C₈ esters into contact with palladium molybdate, so that a change in colour takes place.

The present invention relates to a method for the determination of the content of acrylic acid C₁-C₈ esters in gaseous, combustible gases (fuel gases) and to the use of palladium molybdate for the determination of the content of acrylic acid C₁-C₈ esters in gaseous, combustible gases.

Gas odorization is understood as meaning the addition of odour-intensive substances, which act as warning or alarm substances (odorants), to otherwise odourless gases.

Natural gas chiefly comprises methane (typical methane contents are in the range of 50 to 99 wt. %, usually in the range of 60 to 99 wt. % and conventionally 80 to 99 wt. %) and, depending on its origin, can additionally comprise various contents of ethane, propane and higher molecular weight hydrocarbons. Natural gas H (H=high) has a methane content of from 87 to 99.1 vol. %, natural gas L (L=low) as a rule contains 79.8 to 87 vol. % of methane.

Because of its high purity, the service gas which is used nowadays in the public mains and is conventionally obtained from natural gas is in itself virtually odourless.

If leaks are not noticed promptly, explosive gas/air mixtures with a high hazard potential rapidly build up.

For safety reasons, gas is therefore odorized by addition of odour-intensive substances. Thus, in Germany, for example, the odorization of gases which do not have an adequate intrinsic smell and are distributed in the public gas supply is specified. These odorants are also still perceptible in a high dilution, and because of their exceptionally unpleasant smell, as desired cause an alarm association in humans.

In Germany, about 90% of service gas is currently odorized with tetrahydrothiophene (12-25 mg/m³); in addition, odorization with mercaptans or thioethers is also still conventional.

In the present invention, acrylic acid C₁-C₈ esters are understood as meaning acrylic acid C₁-C₈-alkyl esters, acrylic acid C₂-C₈-alkenyl esters and acrylic acid C₂-C₈-alkynyl esters, in particular acrylic acid C₁-C₈-alkyl esters. In this context, the alkyl, alkenyl and alkynyl radicals can be straight-chain or branched.

In DE-A19837066, the problem of sulfur-free gas odorization was solved by means of mixtures comprising at least one acrylic acid C₁-C₁₂-alkyl ester and a nitrogen compound having a boiling point in the range of 90 to 210° C. and a molecular weight of from 80 to 160, mixtures comprising at least two different acrylic acid alkyl esters being preferred.

A sulfur-free odorant which comprises about 60 wt. % ethyl acrylate, about 37 wt. % methyl acrylate and about 3 wt. % 2,3-methylethylpyrazine and a small amount of an antioxidant is on the market under the name Gasodor (TM) S-Free (TM) (brand name of Symrise GmbH & Co. KG).

The odorant is added to the gas at so-called odorizing stations. This odorized gas is fed to the end consumers via pipelines. At the site of final consumption, the gas must still contain a sufficient amount of the odorant, so that it is ensured that the desired alarm action is caused. On the spot measurements are required in order to check the presence of the necessary minimum odorization. These measurements should take place rapidly and reliably without a high technical outlay, and the measurement results thereby obtained should be unambiguous and conclusive.

The object of the present invention was therefore to develop such a measurement method for the detection of acrylic acid C₁-C₈ esters in gaseous, combustible gases, in particular natural gas. In this context, a direct determination in the fuel gas is to be aimed for, i.e. direct measurement of the odorized gas from the gas pipeline.

U.S. Pat. No. 6,100,097 describes the selective detection of monomeric methyl methacrylate in a liquid, specifically in a liquid with monomers for the production of artificial nails in nail studios. The detection is based on a colour reaction: the complex of palladium molybdate and methyl methacrylate has a blue colour, whereas the complexes of palladium molybdate with other methacrylates are green or yellow in colour. The palladium molybdate reagent is mixed with the monomer-containing liquid to be investigated, the solid complex of palladium molybdate and acrylate(s) formed is filtered off, and this is then washed out with a polar solvent in a container. A blue coloration of the polar solvent after the washing out is said to demonstrate selectively the presence of monomeric methyl methacrylate in the monomer-containing liquid. However, this method does not render possible a quantitative or semi-quantitative conclusion in respect of the content of methyl methacrylate.

Test tubes (measuring tubes) which render possible substance (class)-specific chemisorptive measurements, typically for use in analysis of air, are commercially obtainable. Such test tubes are known, for example, from EP 201 663.

Such test tubes are available, for example, from Dräger, Gastec, Kitagawa or MSA Auer.

The measurement method with test tubes is typically carried out by a procedure in which a precisely defined volume of air to be measured is sucked through the test tube by means of a gas detector pump (air feed pump), as described, for example, in EP 225 520 or U.S. Pat. No. 4,554,133. The test tube is filled with a reagent which is specific for the substance to be determined and changes colour on reaction with the substance. In this context, the length of the reaction zone, i.e. the length of the change in colour, is a measure of the concentration of the substance to be determined. The concentration can be read off, for example, with the aid of a scale attached to the test tube. The gas volume, i.e. the number of strokes to be carried out, must be chosen according to the measurement value to be expected.

Test tubes for the determination of optionally unsaturated esters are commercially obtainable. The commercially obtainable test tubes contain various reagents, at least one of the following disadvantages occurring in the determination of acrylic acid methyl and ethyl ester in natural gas:
a) the reaction takes too long (order of magnitude: 60-120 minutes), the reaction zone in which the change in colour takes place is too small in the case of a shorter measurement time to be able to give a reliable conclusion as to the concentration of acrylates;
b) the change in colour is difficult to recognize;
c) after the change in colour has taken place, the colour becomes paler or changes, which is problematic with slow reactions in particular.

It can additionally be a disadvantage here that several changes in colour take place in succession, so that a reliable evaluation of the measurement result is made difficult.

These test tubes are not suitable for direct connection to a fuel gas pipeline. Since in the present case no determination of acrylic acid C₁-C₈ esters from air (but from fuel gas, e.g. natural gas) is to take place, methods using a gas detector pump are unsuitable.

Many of the reagents employed hitherto for the analysis are based on oxidation reactions of the substance to be measured, the reagent usually being based on a transition metal in a high oxidation state, such as, for example, Cr(VI) or Mn(VII). Since these reagents are comparatively unselective, the presence of other substances can lead to falsification of the measurement results.

For the abovementioned reasons, these methods and the commercially available test tubes are not suitable for the direct determination of acrylic acid C₁-C₈ esters in gaseous, combustible gases.

A method according to the invention for the determination of the concentration of acrylic acid C₁-C₈ esters in fuel gas comprises the following step: bringing the fuel gas (e.g. that is to say natural gas) containing acrylic acid C₁-C₈ esters into contact with palladium molybdate, so that a change in colour takes place.

According to the invention, palladium molybdate is used as the reagent. In this reagent, an easily recognizable change in colour occurs on reaction with acrylic acid C₁-C₈ esters, and in particular a change in colour from pale yellow to blue takes place. The change in colour is probably based on the complexing of the palladium molybdate with the acrylic acid C₁-C₈ esters. This easily recognizable change in colour also takes place during investigation for the presence of acrylic acid C₁-C₈ esters in gaseous, combustible gases having a methane content of at least 60 wt. %.

The palladium molybdate can be used in a pure form, but application to inert carrier materials is preferred. Advantageous carrier materials are, for example, aluminium oxides and silicon oxides. It is also possible to use other carrier materials, such as, for example, aluminium silicates, magnesium oxide, barium sulfate, calcium carbonate and calcium oxide, as well as inert organic carrier materials. Nevertheless, it is to be ensured that the change in colour is not adversely influenced, falsified or even made unreadable by the carrier material.

Preferably, the palladium molybdate is arranged along a reaction zone and the fuel gas containing the acrylic acid C₁-C₈ esters is passed along the reaction zone over the palladium molybdate and brought into contact with this such that the change in colour progresses in the direction of flow of the fuel gas.

In this context, the palladium molybdate is advantageously arranged along the reaction zone and the fuel gas containing the acrylic acid C₁-C₈ esters is passed along the reaction zone over the palladium molybdate and brought into contact with this such that, at least in sections, an amount of 10⁻⁷ mol of acrylic acid C₁-C₈ esters contained in the fuel gas causes a change in colour per 1 cm of length of the reaction zone.

The palladium molybdate can advantageously be on a carrier material, and is then advantageously employed as a powder, particles, grains or granules. The application to the carrier can be carried out, for example, by steeping, precipitation or impregnation.

It is also possible and, where appropriate, advantageous to use not palladium molybdate directly as such, but compound systems from which palladium molybdate can form, such as, for example, (a) palladium sulfate and ammonium molybdate or (b) palladium chloride and lithium molybdate.

The method according to the invention is advantageous in particular for the determination of acrylic acid C₁-C₈-alkyl esters. In this context, the acrylic acid C₁-C₈-alkyl esters are advantageously chosen from the group consisting of:

acrylic acid methyl ester, acrylic acid ethyl ester, acrylic acid n-propyl ester, acrylic acid iso-propyl ester, acrylic acid n-butyl ester, acrylic acid iso-butyl ester, acrylic acid tert-butyl ester, acrylic acid n-pentyl ester, acrylic acid iso-pentyl ester and acrylic acid n-hexyl ester.

The method according to the invention is preferred for the determination of acrylic acid C₁-C₄-alkyl esters, in particular acrylic acid methyl ester, acrylic acid ethyl ester, acrylic acid n-propyl ester, acrylic acid iso-propyl ester, acrylic acid n-butyl ester and acrylic acid iso-butyl ester. Very particularly preferred acrylic acid C₁-C₄-alkyl esters in this respect are acrylic acid methyl ester, acrylic acid ethyl ester and acrylic acid n-butyl ester.

The amount of odorant in the odorized fuel gases, such as are described in DE-A-19837066, is (directly after the odorization) typically in the range of 5-100 mg/m³, preferably 5-50 mg/m³, particularly preferably 10-40 mg/m³ and very particularly preferably 12-30 mg/m³. The method according to the invention is outstandingly suitable for the determination of the concentration of acrylic acid C₁-C₈ esters in such odorized fuel gases.

According to a particularly preferred embodiment of the method according to the invention, the (odorized) fuel gas to be analysed is taken from a stationary fuel gas pipeline and then brought into contact with the palladium molybdate without dilution.

In this case in particular, it is favourable to arrange the palladium molybdate in a measuring tube having an internal diameter in the range of from 0.5 to 5 mm and to pass the fuel gas through the measuring tube.

The invention also relates to a measuring tube comprising palladium molybdate or compounds from which palladium molybdate can be formed, the concentration in the measuring tube, at least in sections, of palladium molybdate or compounds from which palladium molybdate can be formed being chosen such that on reaction thereof with acrylic acid C_{1-C}₈ esters, an amount of 10⁻⁷ mol of acrylic acid C₁-C₈ esters already causes a change in colour per 1 cm of measuring tube length.

Such a measuring tube according to the invention advantageously has an internal diameter in the range of from 0.5 to 5 mm.

The measuring tube according to the invention is outstandingly suitable for the determination of the concentration of acrylic acid C₁-C₈ esters in fuel gas and for use in the methods according to the invention.

The invention is explained in more detail in the following with the aid of examples:

### EXAMPLES

Experiments were carried out under a pipeline overpressure of 22 mbar (smallest known value in gas pipelines; the overpressure value is based on normal pressure of 1,013 mbar), a constant flow of natural gas of approx. 60 ml/min through (a) a measuring tube according to the invention and (b) a commercially available measuring tube being established. The amount of odorant, comprising 60 wt. % ethyl acrylate, 37 wt. % methyl acrylate and 3 wt. % 2,3-methylethylpyrazine, was in each case 18.3 mg/m³ natural gas L.

| | Measuring tube | “Methylacrylat |
|---|---|---|
| | according to the | 5/a” |
| | invention | measuring tube |
| Change in colour | after 10 minutes | after 60 minutes |
| Length of the reaction zone | 2 cm blue | about 1 cm blue |
| Amount of reagent | 50 mg | approx. 1,600 mg |
| (palladium molybdate on a carrier) | | |
| Length of the reagent bed | 2 cm | 7 cm |
| Internal diameter | 2 mm | 7 mm |
| Amount of natural gas L required | 550 ml | 4,000 ml |
| Reading accuracy | good | moderate |
| Sensitivity | high | low |

The “Methylacrylat 5/a” measuring tube is a product of Dräger (Dräger Safety AG & Co. KGa, Lübeck).

So that an interpretable and reliable evaluation of the measurement can take place, the natural gas L odorized with 18.3 mg/m³ must be passed through the commercially obtainable “Methylacrylat 5/a” measuring tube for a period of more than 1 hour. However, since the change in colour to blue formed by the reaction of the ethyl and methyl acrylate with the palladium molybdate reagent already changes again in the direction of grey-black during this period, this measurement is neither exact nor rapid. The use of the commercially obtainable measuring tube is therefore not suitable for the analysis of odorized fuel gas.

So that the shortest possible measurement time can be achieved with a simultaneous reliable quantitative determination of the content of acrylic acid C₁-C₈ esters in the fuel gas, it is advantageous to adhere to certain parameters of the measuring tube according to the invention.

The amount of palladium molybdate reagent (without carrier material), or a corresponding amount of compounds from which the stated amount of palladium molybdate can be formed, in the measuring tube should be in the range of from 5 to 200 mg, preferably in the range of from 10 to 100 mg. The internal diameter of the measuring tube should be in the range of from 0.5 to 5 mm, preferably in the range of from 1 to 4 mm. The length of the reaction zone, i.e. the reagent bed, should be in the range of from 1 to 5 cm, preferably in the range of from 1.5 to 4 cm.

So that a quantification of the odorant can be carried out with the method according to the invention, it should be ensured that a constant stream of natural gas is passed through the measuring tube. The amount of natural gas can be regulated by a pressure or flow regulator.

The best results were achieved with flow regulation after the measuring unit, as is shown in diagram form in the following figure under arrangement 1). Other arrangements can of course also be used, as shown, for example, in arrangements 2and 3).

[image]

[image]

1. Method for the determination of the concentration of acrylic acid C₁-C₈ esters in fuel gas, with the following step:
bringing the fuel gas containing acrylic acid C₁-C₈ esters into contact with palladium molybdate, so that a change in colour takes place.

2. Method according to claim 1, wherein the palladium molybdate is arranged along a reaction zone and the fuel gas containing the acrylic acid C₁-C8 esters is passed along the reaction zone over the palladium molybdate and brought into contact with this such that the change in colour progresses in the direction of flow of the fuel gas.

3. Method according to claim 2, wherein the palladium molybdate is arranged along the reaction zone and the fuel gas containing the acrylic acid C₁-C₈ esters is passed along the reaction zone over the palladium molybdate and brought into contact with this such that, at least in sections, an amount of 10⁻⁷ mol of acrylic acid C₁-C₈ esters contained in the fuel gas causes a change in colour per 1 cm of length of the reaction zone.

4. Method according to claim 1, wherein the fuel gas is natural gas.

5. Method according to claim 1, wherein the fuel gas has a methane content of at least 60 wt. %.

6. Method according to claim 1, wherein the palladium molybdate is applied to a carrier.

7. Method according to claim 1, wherein the fuel gas is taken from a stationary fuel gas pipeline and then brought into contact with the palladium molybdate without dilution.

8. Method according to claim 2, wherein the palladium molybdate is arranged in a measuring tube having an internal diameter in the range of from 0.5 to 5 mm and the fuel gas is passed through the measuring tube.

9. Measuring tube comprising palladium molybdate or compound systems from which palladium molybdate can be formed, the concentration in the measuring tube, at least in sections, of palladium molybdate or compound systems from which palladium molybdate can be formed being chosen such that on reaction thereof with acrylic acid C₁-C₈ esters, an amount of 10⁻⁷ mol of acrylic acid C₁-C₈ esters already causes a change in colour per 1 cm of measuring tube length.

10. Measuring tube according to claim 9, having an internal diameter in the range of from0.5to 5mm.

11. Determining the concentration of acrylic acid C₁-C₈ esters in fuel gas that contains said esters and exhibits a pale yellow color by contacting said fuel gas with palladium molyboate or precursor thereof along a contact length to form a complex that manifests a determinable colour change in said fuel gas.
